(19) United States
(12) Patent Application Publication
(10) Pub. No.: US 2016/0128549 A1
(43) Pub. Date: May 12, 2016

(54) VIDEO ENDOSCOPY SYSTEM, COUPLING DEVICE, VIDEO ENDOSCOPE, AND CONTROL DEVICE

(71) Applicant: OLYMPUS WINTER & IBE GMBH, Hamburg (DE)

(72) Inventor: Thorsten JUERGENS, Hamburg (DE)

(73) Assignee: OLYMPUS WINTER & IBE GMBH, Hamburg (DE)

(21) Appl. No.: 14/987,890

(22) Filed: Jan. 5, 2016

Related U.S. Application Data
(63) Continuation of application No. PCT/EP2014/001671, filed on Jun. 19, 2014.

(30) Foreign Application Priority Data
Jul. 5, 2013 (DE) 102013213247.6

(51) Int. Cl. A61B 1/00; A61B 1/045

(52) U.S. Cl. CPC A61B 1/00126; A61B 1/00006; A61B 1/045; A61B 1/00117

(57) ABSTRACT

A video endoscopy system, including: a video endoscope having an image capturing sensor for recording image data, a controller comprising hardware configured to control the video endoscope, and a coupling for connecting the video endoscope to the controller. The coupling including: a first coupling unit on the video endoscope, a second coupling unit on the controller, an inductive interface between the first and second coupling units for supplying the video endoscope with electrical energy, and an optical interface between the first and second coupling units for transmitting one or more of control data and image data recorded by means of the image capturing sensor.

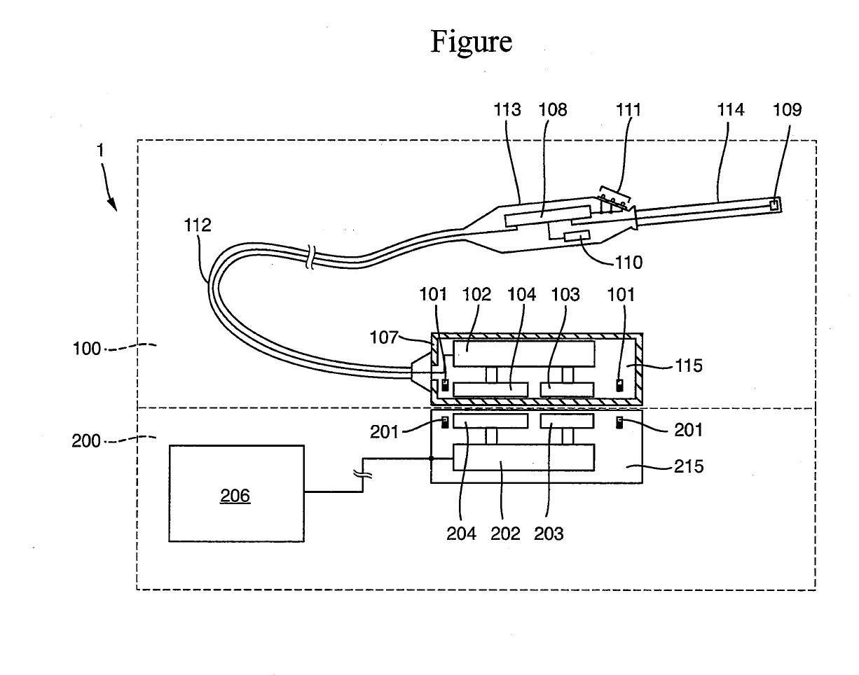

### CROSS-REFERENCE TO RELATED APPLICATION

The present application is a continuation of PCT/EP2014/001671 filed on Jun. 19, 2014, which is based upon and claims the benefit to DE 10 2013 213 247.6 filed on Jul. 5, 2013, the entire contents of each of which are incorporated herein by reference.

### BACKGROUND

1. Field

The present application relates to a video endoscopy system, comprising a video endoscope having an image capturing device for recording image data, a control device for the video endoscope, and a coupling device for connecting the video endoscope to the control device, comprising a first coupling unit on the video endoscope and a second coupling unit on the control device. The present application further provides a coupling device, a video endoscope, and a control device.

2. Prior Art In known generic video endoscopy systems, a releasable connection is generally provided between a video endoscope and a control device. This facilitates the preparation of the video endoscope as well as the transport and storage of the video endoscopy system.

Such video endoscopy systems have galvanic plug-in connections for the power supply of the video endoscope and the signal transmission between the video endoscope and the control device.

Galvanic plug-in connections have the risk of undesired leakage currents and accidents when live plug contacts accidentally come in contact with other conductive parts. Moreover, undesired electromagnetic interference signals are also emitted via the plug-in connections, for example due to impedance jumps.

In order to ensure the electrical operational safety, complex protection mechanisms are necessary, for example a shock protection on the plugs, an insulation of the metallic outer skin of the video endoscope from the electrical inner workings or a corresponding design of the control device with protective switches. Furthermore, complex shielding measures are required so that the appropriate guidelines for electromagnetic compatibility can be met. All of this makes the construction of known video endoscopy systems complex, complicated and expensive.

### SUMMARY

Based on this state of the art, an object is to simplify the construction of a video endoscopy system, wherein the operational safety and the electromagnetic tenability should remain ensured.

Such object is solved by a video endoscopy system, comprising a video endoscope having an image capturing device for recording image data, a control device for the video endoscope, and a coupling device for connecting the video endoscope to the control device, comprising a first coupling unit on the video endoscope and a second coupling unit on the control device, wherein the coupling device is provided with an inductive interface between the two coupling units for supplying the video endoscope with electrical energy, and with an optical interface between the two coupling units for transmitting control data and/or image data recorded by means of the image capturing device.

Within the framework of the present application, a video endoscope is understood as a video endoscope with a distally arranged image capturing device or an endoscope with a separate camera head which has the image capturing device.

The present application provides a releasable connection between the video endoscope and the control device, in which the supply of the endoscope with electrical energy and the data transmission to the control device are ensured without galvanic connections, such as without galvanic plug-in connections. Even without special protective measures, the present application thus offers the advantage of a particularly safe energy supply because no leakage currents and accidents can occur due to open, live plug contacts.

An optical interface is also provided for transmitting image data from the image capturing device to the control device. An optical interface offers an advantage in that it does not transmit electromagnetic radiation in the electrically or electronically relevant frequency range. If the optical interface can be configured bidirectionally, the video endoscope can also be controlled via the optical interface.

The embodiments disclosed in the present application thus ensure to a high degree an electromagnetic compatibility of the video endoscopy system. Furthermore, such embodiments offer a durable, permanent and wear-and-tear-proof releasable connection between the video endoscope and control device. In the disclosed embodiments, there are no plug contacts, the conductivity and thus functionality of which can be impaired or warped due to corrosion or material wear.

One of the two coupling units can be configured as a plug and the other coupling unit as a receiver for the plug, wherein the plug can be arranged on the video endoscope and the receiver for the plug on the control device. An alignment of the components of the inductive interface as well as the optical interface with each other is thereby ensured even without mechanically engaging electrical plug contacts for connection of the video endoscope and the control device and the proper functionality of the interfaces is thus ensured. For this, the plug and the receiver can be configured complementary in shape at least in sections.

In one embodiment, the first coupling unit and/or the second coupling unit can comprise at least one magnet for the releasable connection of the two coupling units. The respective counterpart can be a magnet or a magnetizable element so that a magnetic coupling is achieved, which can also align the inductive and optical interface with each other in a suitable manner.

Damage to the coupling units can thereby be prevented. The magnets or the magnetic holders can be configured such that the coupling units disconnect from each other in the case of an accidental mechanical load on the connection.

Furthermore, a simple shape of the coupling units is enabled, which have for example even contact surfaces facing each other when the connection or coupling is closed. Hard to clean corners or edges on the surfaces of both coupling units can thereby be avoided.

The second coupling unit can comprise a transmitting coil for the inductive interface, in which case the first coupling unit can comprise a receiving coil for the inductive interface.

For example, the transmitting coil and the receiving coil can be respectively configured as a cylinder coil or solenoid with a center axis, wherein the center axes of the two coils can be aligned parallel to each other, such as congruent, when the connection or coupling between the video endoscope and the control device is closed. The inductive interface is this aligned.

The inductive interface can advantageously correspond with the Qi standard of the Wireless Power Consortium, Piscataway, N.J., USA.

The transmitting coil can be part of a first oscillating circuit and the receiving coil can be part of a second oscillating circuit, wherein the first oscillating circuit and the second oscillating circuit can have at least one mainly corresponding resonance frequency. Oscillating circuits with coordinated resonances enable for example the maximization of the transmitted power, the optimization of the efficiency of the power transmission and the adjustment of the transmitted power based on demand.

At least one of the two oscillating circuits can be configured with a changeable resonance behavior in order to be able to provide coordination of the two oscillating circuits and to be able to adjust them if necessary.

The first coupling unit can comprise an optical transmitting unit of the optical interface, which can transmit in the infrared spectral range, in which case the second coupling unit can comprise an optical receiving unit of the optical interface. The second coupling unit also can comprise an optical transmitting unit and the first coupling unit an optical receiving unit. The optical interface is thus bidirectional.

Such optical interfaces can operate at a high transmission frequency, for example 50 MHz to 5 GHz, and thereby provide a high transmission bandwidth. Optical interfaces in the infrared spectral range also require a direct line of sight between the transmitting unit and the receiving unit and their functionality is set even without additional optical components such as light conductors, lenses or apertures between the transmitting unit and the receiving unit. If applicable, transparent viewing windows can be provided, such as in the infrared spectral range, for example when the transmitting unit and/or the receiving unit is/are arranged in one or each in one housing that is non-transparent for infrared radiation.

The transmitting unit can be a semi-conductor diode and/or the receiving unit can be a semi-conductor diode. Diodes or semi-conductors are small and can be operated reliably with a long service life. Moreover, they are cheap mass-produced items. The transmitting unit can be, for example, a laser diode. The receiving unit can be, for example, a photo diode.

The video endoscope can comprise a shaft, a cable and a handle arranged between the shaft and the cable, wherein the image capturing device can be arranged on an end of the shaft facing away from the handle and/or the first coupling unit can be arranged on an end of the cable facing away from the handle, wherein the image capturing device and the first coupling unit can be galvanically interconnectable or connected.

The video endoscope for example can thereby be inserted with the shaft into a natural or operatively created body opening of a patient to be examined in order to achieve an observation area within the patient. The handle can thereby remain outside of the patient making it possible for the treating physician to hold the video endoscope and to guide the shaft.

The cable serves as a connection between the handle and the coupling device.

The video endoscope can further comprise a light source for illuminating an observation area of the image capturing device. The light source can thereby be supplied with electrical energy by means of the inductive interface of the coupling device. The light source can be configured as a light-emitting diode or LED. Light-emitting diodes are small, light and cheap and energy-efficient. The light source can be arranged on or in a handle of the video endoscope or on a distal end of a shaft of the video endoscope.

The video endoscope can be configured in an autoclavable manner, wherein a shaft, a handle and a cable of the video endoscope as well as the first coupling unit can be combined into an autoclavable structural unit. Within the framework of the present disclosure, autoclaving means, for example, preparing with superheated steam under hyperbaric conditions.

For this, all components of the assembly can be enclosed in an uninterrupted hermetic barrier, such as a temperature-resistant and steam-tight plastic coating.

The control device can comprise a display unit for representing image data captured by the image capturing device, wherein the display unit can be connectable or connected galvanically with the second coupling unit.

The display unit can be configured as a screen or monitor and can be galvanically connected or connectable with the second coupling unit such as by means of a permanent, for example soldered, cable connection or releasably by means of a plug-in connection.

The object can be further solved by a coupling device of the previously described video endoscopy system, wherein the coupling device comprises a first coupling unit for a video endoscope and a second coupling unit for a control device, wherein the coupling device is provided with an inductive interface and an optical interface between the two coupling units.

The object can be further solved by a video endoscope of a video endoscopy system, wherein the video endoscope comprises an image capturing device and a first coupling unit of a coupling device.

The object can be further solved by a control device of a video endoscopy system, wherein the control device comprises a second coupling unit of a coupling device.

Further characteristics will become evident from the description of the embodiments together with the claims and the attached drawing. Embodiments can fulfill individual characteristics or a combination of several characteristics.

### BRIEF DESCRIPTION OF THE DRAWINGS

Without restricting the general idea of the invention, the invention is described below on the basis of exemplary embodiments with reference to the drawings, whereby reference is explicitly made to the drawings with regard to all the details, which are not described in more detail in the text. In the figures:

The FIGURE is an exemplary schematic illustration of a video endoscopy system.

In the FIGURE, the same or similar elements and/or parts are provided with the same reference numbers in order to prevent the item from needing to be reintroduced.

### DETAILED DESCRIPTION

The FIGURE shows schematically an example of a video endoscopy system 1 with a video endoscope 100 and a control device 200 for the video endoscope 100. The video endoscope 100 and the control device 200 are interconnected in a releasable manner by means of a plug 115 on the video endoscope 100 and a socket 215 on the control device 200.

The video endoscope 100 comprises a shaft 114, which is connected with a handle 113 on the proximal end. The shaft 114 can be configured to be guided to an observation or examination area inside a patient body through natural or artificial, such as operatively created, openings.

An image capturing device 109 comprising for example a CCD chip is located on the distal end of the shaft 114, such as opposite the handle 113, in order to record an image from the observation area and to convert it to electrical signals.

Handle electronics 108 connected with the image capturing device 109 for reading electrical signals from the image capturing device 109 as well as signal conditioning and for signal integration, if applicable also for processing control signals, is located in the handle 113 of the video endoscope 100.

The handle electronics 108 is also connected with a light-emitting diode 110 for illuminating an observation area of the video endoscope 100. For this, the light-emitting diode 110 is arranged for example in the handle 113, wherein optical fibers or light-emitting diodes (not shown) direct the light of the light-emitting diode 110 to the observation area on the distal end of the shaft 114. Alternatively, the light-emitting diode 110 can be arranged on or in the shaft 114, such as on the distal end of the shaft 114, and illuminates the observation area directly.

For controlling the handle electronics 108, the image capturing device 109 and/or the light-emitting diode 110, push buttons 111, for example for switching the light-emitting diode 110 and/or the image capturing device 109 on and off, are also provided on the handle 113 of the video endoscope.

The handle 113 is connected with a plug 115 by means of a cable 112 on the back side, such as on the side facing away from the shaft 114. At least one electrical cable, which connects the handle electronics 202 galvanically with the plug 115, such as the plug electronics 102 arranged in the plug, is guided through the cable 112. Moreover, the cable 112 can have an electromagnetic shielding (not shown).

The shaft 114, the handle 113, the cable 112 and the plug 115 can be respectively configured in a temperature-resistant and pressure-resistant manner. Such named components can be heated to over 130° C. multiple times without being damaged in a pressurized atmosphere of at least 3 bars of excess pressure. Furthermore, such named components can be combined into one assembly and surrounded with a steam-impermeable and temperature-resistant barrier in the form of a casing with a suitable plastic. The entire endoscope 100 can be thereby autoclavable, i.e. it can be sterilized by superheated steam in a hyperbaric autoclave.

The control device 200 comprises a socket 215 and a display unit 206 connected galvanically with the socket 215, for example in the form of a monitor or screen, for displaying an image or video, which can be obtained in real time from the image data from the image capturing device 109.

The plug 115 and the socket 215 each have magnets 101, 201 in order to establish a releasable connection between the plug 115 and the socket 215 or respectively between the video endoscope 100 and the control device 200 and to hold the plug 115 in a desired position relative to the socket 215.

Additionally or alternatively, the plug 115 and the socket 215 can be configured complementary in shape at least in sections so that plug 115 and socket 215 are interconnectable or connected mechanically in a releasable manner, for example by means of a clamp connection.

An inductive interface 104, 204 is provided between plug 115 and socket 215 in order to supply the video endoscope 100 with electrical energy. For this, the socket 215 has a transmitting coil 204 and the plug 115 has a receiving coil 104.

The transmitting coil 204 is part of a first oscillating circuit, which, in addition to the transmitting coil 204, comprises at least parts of electronics 202 of the socket 215. In a comparable manner, the receiving coil 104 is part of a second oscillating circuit, which, in addition to the receiving coil 104, comprises at least parts of plug electronics 102. The oscillating circuits each have at least one resonance coordinated for the respective other oscillating circuit. At least one of the two oscillating circuits can be configured with a changeable or adjustable resonance behavior, for example by means of at least one trimmable capacitor.

The plug electronics 102 and the electronics 202 of the socket 215 also have circuits for monitoring the transmission of electrical energy or electrical power by means of the inductive interface 104, 204.

Furthermore, an optical interface 103, 203 is provided between the plug 115 and the socket 215 for communication or for the exchange of data, such as for transmitting image data from the image capturing device 109 to the display unit 206, between the video endoscope 100 and the control device 200. For this, the plug 115 has an infrared-light-emitting semi-conductor diode 103, configured for example as a laser diode, and the socket 215 can have a photo diode 203, sensitive, for example, to infrared light.

The laser diode 103 is connected with the plug electronics 102, wherein the plug electronics comprises circuits for monitoring the communication with the control device 200 by means of the laser diode 103.

The photo diode 203 is connected with the electronics 202 of the socket 215, wherein the electronics 202 comprises circuits for monitoring the communication with the video endoscope 100 by means of the photo diode 203.

The optical interface 103, 203 can be operated by means of amplitude modulations with a modulation frequency between 50 MHz and 5 GHz, whereby transmission bandwidths in the range of several Gbit/s are achievable.

The plug 115 has a housing 107, which is non-transparent or partially non-transparent for infrared radiation in the example shown in the FIGURE. A window transparent for infrared radiation is thus provided in order to guarantee communication between laser diode 103 and photo diode 203. The window must thereby only be transparent for infrared radiation so that non-transparent plastics can be used as the material for the window also in the visible spectral range.

Other components of the control device 200 are not shown in the FIGURE for the sake of clarity.

For example, signal electronics is provided between electronics 202 of the socket and the display unit 206 in order to further edit or process the image data from the image generating device 109 received via the optical interface 103, 203 and to prepare it for display.

Furthermore, the control device 200 can have further operating and monitoring elements with corresponding electronics for monitoring individual functions of the control device 200 and/or of the video endoscope 100, for example an adjustment option for the brightness of the light-emitting diode 110 or the sensitivity of the image generating device 109.

For this, potentially required control signals can be transmitted from the control device 200 to the video endoscope 100 by means of the inductive interface 103, 203, for example by means of a superimposed amplitude modulation.

Alternatively or additionally, the optical interface 103, 203 can be configured for the bidirectional communication between video endoscope 100 and control device 200. For this, for example the socket 215 also has a light-emitting semi-conductor diode 103 and the plug 115 also has a photo diode 203.

All named characteristics, including those taken from the drawings alone and also individual characteristics which are disclosed in combination with other characteristics are considered alone and in combination as essential. Embodiments can be realized by individual characteristics or a combination of several characteristics.

### REFERENCE LIST

1 Video endoscopy system

100 Video endoscope

101 Magnet

102 Electronics

103 IR laser diode

104 Receiving coil

106 Viewing window

107 Housing

108 Electronics

109 Image capturing device

110 Light-emitting diode

111 Push button

112 Cable

113 Handle

114 Shaft

200 Control device

201 Magnet

202 Electronics

203 IR photo diode

204 Transmitting coil

206 Display unit

What is claimed is:

1. A video endoscopy system, comprising:
a video endoscope having an image capturing sensor for recording image data,
a controller comprising hardware configured to control the video endoscope, and
a coupling for connecting the video endoscope to the controller, the coupling comprising:
a first coupling unit on the video endoscope,
a second coupling unit on the controller,
an inductive interface between the first and second coupling units for supplying the video endoscope with electrical energy, and
an optical interface between the first and second coupling units for transmitting one or more of control data and image data recorded by means of the image capturing sensor.

2. The video endoscopy system according to claim 1, wherein one of the first and second coupling units is configured as a plug and an other of the first and second coupling units is configured as a receiver for the plug.

3. The video endoscopy system according to claim 2, wherein the plug is arranged on the video endoscope and the receiver for the plug is arranged on the controller.

4. The video endoscopy system according to claim 1, wherein one or more of the first coupling unit and the second coupling unit comprise at least one magnet to configure a releasable connection between the first and second coupling units.

5. The video endoscopy system according to claim 1, wherein the inductive interface comprising the second coupling unit having a transmitting coil and the first coupling unit having a receiving coil.

6. The video endoscopy system according to claim 5, wherein the transmitting coil is part of a first oscillating circuit and the receiving coil is part of a second oscillating circuit.

7. The video endoscopy system according to claim 6, wherein the first oscillating circuit and the second oscillating circuit have at least one mainly corresponding resonance frequency.

8. The video endoscopy system according to claim 1, wherein the optical interface comprises one of the first and second coupling units having an optical transmitting unit and an other of the first and second coupling units having an optical receiving unit

9. The video endoscopy system according to claim 8, wherein the first coupling unit having the optical receiving unit and the second coupling unit having the optical transmitting unit.

10. The video endoscopy system according to claim 8, wherein the optical transmitting unit is configured to transmit in an infrared spectral range.

11. The video endoscopy system according to claim 8, wherein one or more of the optical transmitting unit and the optical receiving unit is configured as a semi-conductor diode.

12. The video endoscopy system according to claim 11, wherein one or more of the optical transmitting unit is configured as a laser diode and the optical receiving unit is configured as a photo diode.

13. The video endoscopy system according to claim 1, wherein the video endoscope further comprising:
a shaft having the image capturing sensor,
a cable for electrically connecting the image capturing sensor to the controller, and
a handle arranged between the shaft and the cable.

14. The video endoscopy system according to claim 13, wherein the image capturing sensor is arranged on an end of the shaft facing away from the handle and the first coupling unit is arranged on an end of the cable facing away from the handle and the image capturing sensor and the first coupling unit are galvanically connected.

15. The video endoscopy system according to claim 1, wherein the video endoscope further comprises a light source for illuminating an observation area of the video endoscope wherein the light source is arranged on one of a handle of the video endoscope and on a distal end of a shaft of the video endoscope.

16. The video endoscopy system according to claim 15, wherein the light source comprises a light-emitting diode.

17. The video endoscopy system according to claim 1, wherein the video endoscope is configured in an autoclavable manner.

18. The video endoscopy system according to claim 17, wherein a shaft, a handle and a cable of the video endoscope as well as the first coupling unit are combined into an autoclavable structural unit.

19. A coupling for a video endoscopy system, the coupling comprising:
a first coupling unit for a video endoscope; and
a second coupling unit for a controller connectable to the video endoscope,
wherein the coupling having an inductive interface and an optical interface between the first and second coupling units.

20. A video endoscope of a video endoscopy system, the video endoscope comprising:
an image capturing sensor; and
a coupling for connecting to a mating coupling for a controller, the coupling of the video endoscope comprising:
one of a transmitting coil and a receiving coil for configuring an inductive interface between the image capturing sensor and the controller; and
one of an optical transmitting unit and optical receiving unit for configuring an optical interface between the image capturing sensor and the controller.

21. A control device for a video endoscopy system, the control device comprising:
a controller comprising hardware; and
a coupling for connecting to a mating coupling for a video endoscope, the coupling of the control device comprising:
one of a transmitting coil and a receiving coil for configuring an inductive interface between the video endoscope and the controller; and
one of an optical transmitting unit and optical receiving unit for configuring an optical interface between the video endoscope and the controller.
